(19) United States
(12) Patent Application Publication
(10) Pub. No.: US 2009/0054513 A1
(43) Pub. Date: Feb. 26, 2009

(54) METHOD OF MANAGING BLOOD GLUCOSE LEVELS, INSULIN LEVELS AND/OR INSULIN RECEPTOR FUNCTIONALITY IN INDIVIDUALS WITH DIABETES, POLYCYSTIC OVARIAN SYNDROME AND/OR ALZHEIMER'S DISEASE

(75) Inventors: Gregory D. WEBSTER, Orlando, FL (US); Emmanuel C. OPARA, Durham, NC (US)

Correspondence Address: HOGAN & HARTSON LLP, ONE TABOR CENTER, SUITE 1500, 1200 SEVENTEENTH ST, DENVER, CO, 80202, US

(73) Assignee: RESPONSE SCIENTIFIC, INC., ORLANDO, FL (US)

(21) Appl. No.: 11/855,808

(22) Filed: Sep. 14, 2007

Related U.S. Application Data
(63) Continuation-in-part of application No. 11/843,525, filed on Aug. 22, 2007.

(51) Int. Cl. A61K 31/385; A61P 15/00; A61P 3/08

(52) U.S. Cl. 514/440

(57) ABSTRACT

A combination of constituents for oral administration by women with polycystic ovarian syndrome includes α-lipoic acid, linolenic acid complex, biotin, and coenzyme Q-10. A preferred method of manufacture is by separate microencapsulation of one or more of the components followed by encapsulation of the individual components, for oral administration. Other methods of delivery include packaging in impermeable, disposable packets and mixing the formulations with food or a cold liquid. A combination of constituents for administration by either men or women to encourage increase in brain insulin levels and/or brain insulin receptor functionality also includes α-lipoic acid, linolenic acid complex, biotin, and coenzyme Q-10.

### RELATED APPLICATION

The present application is a continuation-in-part application of co-pending U.S. Ser. No. 11/843,525 filed Aug. 22, 2007, which is incorporated herein in its entirety by this reference.

### FIELD OF THE INVENTION

The present invention relates to use of compositions to manage blood glucose levels, insulin levels and/or insulin receptor functionality in individuals with diabetes, polycystic ovarian syndrome and/or Alzheimer's disease.

Background of the Invention

Polycystic ovarian syndrome (referred to herein as “PCOS”) is also known as Stein-Leventhal Syndrome, affects 6-10% of women and is a leading cause of infertility in women. Symptoms include irregular menstrual cycles, ovarian cysts, high blood pressure, acne, elevated insulin levels, insulin resistance, diabetes, excess facial and body hair, alopecia and obesity centered around the midsection. Although PCOS was previously and is sometimes referred to as polycystic ovarian disease, its cause is widely recognized as not conclusively identified, and so may be thought of as a group of related symptoms or classified more generally as an endocrine disorder.

One group of PCOS symptoms involves insulin imbalance and/or resistance. A theory regarding hyperinsulinemia as a symptom of PCOS is that the hyperinsulinemia results not from excess production of insulin by beta cells in the pancreas, but rather from excess production of insulin elsewhere in the body. Accordingly, conventional medications such as Metformin aimed at managing type 2 diabetes and excess insulin produced by the pancreas in response to hyperglycemia may not be fully effective in treating hyperinsulinemia in women with PCOS. In any case, and however characterized, PCOS can result in an inability to conceive.

Individuals with Alzheimer's disease are also reported to exhibit insulin production and/or receptivity issues. In an article entitled Alzheimer's Disease May be ‘Type 3’ Diabetes, at http://health.dailynewscentral.com/content/view/0001969/53/, it is reported that insulin levels and insulin production in the brain decrease as Alzheimer's disease advances. Further, insulin-related growth factor-I loses its ability to bond to cell receptors, causing resistance to insulin growth factors. Accordingly, insulin receptor functionality descreases.

Diabetes mellitus includes diabetes mellitus types 1 and 2. Diabetes mellitus type 2 (sometimes referred to as diabetes mellitus type II and adult-onset diabetes) is a metabolic disorder typically involving insulin resistance, in which the cells of the body of an individual do not respond appropriately when insulin is present. If unnoticed or left untreated, severe complications can result, including renal failure, blindness and wounds that fail to heal. While there is an inheritable genetic connection, more than 80% of the individuals with diabetes type 2 are overweight or obese. Diabetes mellitus type 1 usually results from an autoimmune disorder that destroys pancreatic beta cells which produce insulin.

Metformin (1-(diaminomethylidene)-3,3-dimethyl-guanidine) is an anti-diabetic drug having the formula C₄H₁₁N₅, and is available by prescription under the trade names Glucophage™, Diabex™, Diaformin™ and others. General forms are also available. Metformin appears to reduce hepatic gluconeogenesis, decrease absorption of glucose from the gastrointestinal tract and increase insulin sensitivity. Adverse effects include impaired liver or kidney function, diarrhea, cramps, nausea, vomiting, mal-absorption of vitamin B12 and possible B12 deficiency. Metformin is available in immediate release formulations of 500 mg., 850 mg., and 1000 mg. tablets and in slow and extended release formulations of 500 mg. and 750 mg.

Metformin is often prescribed with rosiglitazone, one form of which is marketed under the trade name Avandia®. While Avandia® has been approved by the Food & Drug Administration (FDA) to treat diabetes mellitus, the FDA recently issued a safety alert on Avandia®, stating that
Safety data from controlled clinical trials have shown that there is a potentially significant increase in the risk of heart attack and heart-related deaths in patients taking Avandia. However, other published and unpublished data from long-term clinical trials of Avandia, including an interim analysis of data from the RECORD trial (a large, ongoing, randomized open label trial) and unpublished reanalyses of data from DREAM (a previously conducted placebo-controlled, randomized trial) provide contradictory evidence about the risks in patients treated with Avandia.
Patients who are taking Avandia, especially those who are known to have underlying heart disease or who are at high risk of heart attack should talk to their doctor about this new information as they evaluate the available treatment options for their type 2 diabetes.
FDA's analyses of all available data are ongoing. FDA has not confirmed the clinical significance of the reported increased risk in the context of other studies . . .

For some patients, the uncertainly of such risks, as well as problems associated with long-term use of Metformin (e.g., need for increased dosages over time), results in an ongoing search for alternatives to address symptoms and underlying physiological conditions related to diabetes mellitus.

Metformin is also prescribed with Amaryl®, available from Sanofi-Aventis and also generically available as glimepiride. Amaryl® is a long-acting, III generation sulfonylurea: 3-ethyl-N,N-bis (3-ethyl-4-methyl-2-oxo-5H-pyrrol-2-yl)-4-methyl-2-oxo-5H-pyrrole-1-carboxamide. Glimepiride lowers blood glucose levels by stimulating pancreatic beta cells to produce more insulin and by inducing increased activity of peripheral insulin intracellular receptors. However, gastrointestinal disturbance can result.

Lantus®, an insulin analogue used to help control blood sugar levels, is prescribed to complement the shorter-acting sulfonylurea drugs. Lantus® is characterized as having a 24-hour duration of action, thereby resembling basal insulin secretion of pancreatic beta cells and minimizing nocturnal hypoglycemia. However, Lantus® typically requires the support of a fast acting insulin taken with food to reduce the effect of meal-derived increase in blood glucose levels.

Exenatide, marketed under the trade name Byetta® and available from Eli Lilly and Company, constitutes a new class of medications approved for treating diabetes. Exenatide is a peptide containing 39 amino acids which functions as an insulin secretagogue and has glucose regulating capabilities. Exenatides are often combined with Metformin and sulfonylureas to improve glucose control. However, exenatides do have some adverse qualities, e.g., they require administration by injection and cause gastrointestinal disturbances in some patients. Exenatide may also increase risk of sulfonylurea-induced hypoglycemia.

Thus, while the above drug therapies, alone and in combination with each other and with other drugs provide significant and life-extending relief from diabetes mellitus, typically, over time, dosages must be increased and new combinations of drugs tried for an individual with diabetes mellitus to maintain acceptable blood glucose levels and a satisfactory life style. As increased dosages and/or combinations are prescribed, treatment costs may increase, the presence of side effects may become manifest, and administration by injections (as compared to oral regimens) may be required. For these reasons, use of nutritional supplements to prevent or control diabetes mellitus has been explored.

U.S. Pat. No. 6,203,819 entitled Dietary Supplement and Method of Treatment for Diabetic Control, discloses a daily nutritional supplement to assist in the metabolism of glucose. So-called “anchor components” include chromium polynicotinate, picolinate, vanadyl sulfate, vitamin E natural, standardized willow bark (as a source of aspirin), magnesium chloride, citrate, fumarate, malate, glutorate, and succinate complex, folic acid and alpha-lipoic acid. This nutritional supplement is more succinctly described in the Summary of the Invention as comprising effectives amounts of sources of chromium, vanadium, magnesium, vitamin E, aspirin, folic acid and alpha-lipoic acid. Essential components claimed include chromium, vanadium and aspirin. However, vanadyl sulfate has been reported to cause gastrointestinal distress and there remains some question about disposition of vanadium in the body after long-term ingestion.

U.S. Pat. No. 6,585,998 entitled Nutraceutical Composition, relates to a nutraceutical composition which is used to maintain normal blood sugar levels and normal levels of non-enzymatic protein glycosylation. The composition requires at least 7 constituents: a tripeptide component, guanidine hydrochloride, alpha-lipoic acid, a brazilin component, an amino acid component, a flavonoid component and a catalase. The addition of selenium is also suggested.

The extent to which the above-described treatments and supplements are useful in managing blood glucose levels in individuals generally and pre-diabetic individuals in particular, varies by individual and over a course of a lifetime in such individuals. The precise extent to which such treatments may have an impact on a woman diagnosed with PCOS has not been fully measured.

U.S. Patent Publication No. 2007/0203134 A1 entitled Benzimidazole Acetonitriles, claims pharmaceutical formulations of benzimidazole acetonitriles useful in the treatment of metabolic disorders mediated by insulin resistance or hyperglycemia, including PCOS. The publication identifies and describes the manufacture of numerous chemical structures as applicable to the invention. A test involving 24 mice is described as determining the anti-diabetic effect of a formula (I), in which blood glucose levels induced by food intake decreased by about 20-40%. No tests involving PCOS or infertility effects are reported.

Also by way of example, U.S. Patent Publication No. 2007/0155735 A1 entitled Novel Aminoindazole Derivatives as Medicaments and Pharmaceutical Compositions Including Them, identifies and describes the manufacture of aminoindazole derivatives as useful Alzheimer's disease, Parkinson's disease, frontoparietal dementia, corticobasal degeneration, Pick's disease, strokes, cranial and spinal traumas and peripheral neuropathies, obesity, metabolic diseases, type II diabetes, essential hypertension, atherosclerotic cardiovascular diseases, polycystic ovaries syndrome, syndrome X, immunodeficiency and cancer. No tests involving control of blood glucose levels generally or PCOS or infertility effects specifically are reported.

By way of further example, U.S. Patent Publication No. 2007/0037826 A1 entitled Indolylmaleimide Derivatives, identifies and describes the manufacture of indolylmaleide derivatives and suggests they are useful which are useful in the treatment and/or prevention of diseases or disorders mediated by T lymphocytes and/or PKC, e.g. acute or chronic rejection of organ or tissue allo- or xenografts, graft versus host diseases, atherosclerosis, vascular occlusion due to vascular injury such as angioplasty, restenosis, obesity, syndrome X, impaired glucose tolerance, polycystic ovary syndrome, hypertension, heart failure, chronic obstructive pulmonary disease, CNS diseases such as Alzheimer disease or amyotrophic lateral sclerosis, cancer, infectious diseases such as AIDS, septic shock or adult respiratory distress syndrome, ischemia/reperfusion injury e.g. myocardial infarction, stroke, gut ischemia, renal failure or hemorrhage shock, or traumatic shock, e.g. traumatic brain injury. No tests involving control of blood glucose levels generally or PCOS or infertility effects specifically are reported.

U.S. Patent Publication No. US 2007/0009608 entitled Composing Comprising Plant and/or Fish Oils and Compounds Comprising Non-Oxidizable Fatty Acid Entities claims a method of prevention and/or treatment of numerous conditions, including insulin resistance, polycystic ovary syndrome and cancer, as follows:
A method of prevention and/or treatment of insulin resistance, obesity, diabetes, fatty liver, hypercholesterolemia, dyslipidemia, atherosclerosis, coronary heart disease, thrombosis, stenosis, secondary stenosis, myocardial infarction, stroke, elevated blood pressure, endothelial dysfunction, procoagulant state, polycystic ovary syndrome, the metabolic syndrome, cancer, an inflammatory disorder, and a proliferate skin disorder comprising the administration of a pharmaceutical or nutritional composition comprising a combination of:
1) plant oil and/or fish oil; and
2) one or more compounds comprising non β-oxidizable fatty acid entities represented by
(a) the general formula R″—COO—(CH₂)₂ₙ₋₁—X—R′, wherein X is a sulphur atom, a selenium atom, an oxygen atom, a CH₂ group, a SO group or a SO₂ group; n is an integer of 0 to 11; and R′ is a linear or branched alkyl group, saturated or unsaturated, optionally substituted, wherein the main chain of said R′ contains from 13 to 23 carbon atoms and optionally one or more heterogroups selected from the group comprising an oxygen atom, a sulphur atom, a selenium atom, an oxygen atom, a CH₂ group, a SO group and a SO₂ group, and R″ is a hydrogen atom or an alkyl group containing from 1 to 4 carbon atoms; and/or
(b) the general formula (I),

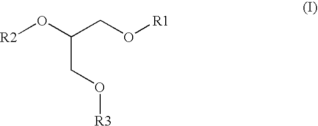

wherein R1, R2, and R3 represent
i) a hydrogen atom; or
ii) a group having the formula CO—R in which R is a linear or branched alkyl group, saturated or unsaturated, optionally substituted, and the main chain of said R contains from 1 to 25 carbon atoms; or
iii) a group having the formula CO—(CH₂)₂ₙ₊₁—X—R′, wherein X is a sulphur atom, a selenium atom, an oxygen atom, a CH₂ group, a SO group or a SO₂ group; n is an integer of 0 to 11; and R′ is a linear or branched alkyl group, saturated or unsaturated, optionally substituted, wherein the main chain of said R′ contains from 13 to 23 carbon atoms and optionally one or more heterogroups selected from the group comprising an oxygen atom, a sulphur atom, a selenium atom, an oxygen atom, a CH₂ group, a SO group and a SO₂ group;
iv) an entity selected from the group comprising —PO₃CH₂CHNH₃COOH (serine), PO₃CH₂CH₂NH₃ (ethanolamine), PO₃CH₂CH₂N(CH₃)₃ (choline), PO₃CH₂CHOHCH₂OH (glycerol) and PO₃(CHOH)₆ (inositol);
wherein R1, R2, and R3 are chosen independently from i), ii), iii), or iv), but at least one of R1, R2, or R3 is defined by iii); and/or
(c) the general formula (II),

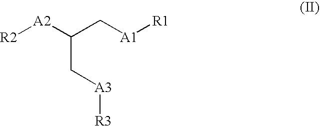

wherein A1, A2 and A3 are chosen independently and represent an oxygen atom, a sulphur atom or an N—R4 group in which R4 is a hydrogen atom or a linear or branched alkyl group, saturated or unsaturated, optionally substituted, containing from 1 to 5 carbon atoms;
wherein R1, R2, and R3 represent
i) a hydrogen atom or a linear or branched alkyl group, saturated or unsaturated, optionally substituted, containing from 1 to 23 carbon atoms; or
ii) a group having the formula CO—R in which R is a linear or branched alkyl group, saturated or unsaturated, optionally substituted, and the main chain of said R contains from 1 to 25 carbon atoms; or
iii) a group having the formula CO—(CH₂)₂ₙ₊₁—X—R′, wherein X is a sulphur atom, a selenium atom, an oxygen atom, a CH₂ group, a SO group or a SO₂ group; n is an integer of 0 to 11; and R′ is a linear or branched alkyl group, saturated or unsaturated, optionally substituted, wherein the main chain of said R′ contains from 13 to 23 carbon atoms and optionally one or more heterogroups selected from the group comprising an oxygen atom, a sulphur atom, a selenium atom, an oxygen atom, a CH₂ group, a SO group and a SO₂ group;
iv) an entity selected from the group comprising —PO₃CH₂CHNH₃COOH (serine), PO₃CH₂CH₂NH₃ (ethanolamine), PO₃CH₂CH₂N(CH₃)₃ (choline), PO₃CH₂CHOHCH₂OH (glycerol) and PO₃(CHOH)₆ (inositol);
wherein R1, R2, and R3 are chosen independently from i), ii), iii), or iv), but at least one of R1, R2, or R3 is defined by iii); and/or
a salt, prodrug or complex of the compounds according to (a)-(c).

Clearly, many newly developed compounds are suggested to be useful as pharmaceutical formulations for the treatment of insulin resistance, diabetes and PCOS, and a host of other diseases, but evidence of actual treatment of PCOS or increase in fertility has not been demonstrated in the above patent application publications. However, evidence of actual treatment of PCOS or increase in fertility from the precise, complex fish oil mixtures is not yet demonstrated.

Accordingly, there remains a need for a simplified medical food or nutritional supplement effective to manage blood glucose levels in individuals generally and women suffering from PCOS or infertility specifically.

### SUMMARY OF THE INVENTION

The present invention, which relates to compositions, combinations of constituents thereof, medical foods and nutritional supplements useful for controlling blood glucose levels, insulin levels and insulin receptor functionality (i) in women so as to alleviate symptoms of polycystic ovarian syndrome (PCOS), and (ii) in adults generally so as to obviate the development or progression of Alzeimer's disease, includes alpha lipoic acid (“α-lipoic acid”), linolenic acid complex, biotin and coenzyme Q-10. Acceptable ranges of the four constituents per day of the preferred formulation of the present invention are as follows:
α-lipoic acid-100 to 2500 mg.;
linolenic acid complex-10 to 4000 mg.;
biotin-2 to 25 mg.; and
coenzyme Q-10-25 to 500 mg.

A preferred method of alleviating symptoms of PCOS and/or increasing fertility in women with PCOS involves the management of blood glucose levels, insulin levels, and insulin receptor functionality by ingestion of the formulations of the present invention. This method is also applicable to adults generally to obviate development or progression of Alzheimer's disease.

A preferred method of manufacturing the compositions, medical foods and nutritional supplements of the present invention is to microencapsulate each component, then assemble the microencapsulated components for collective oral administration, for example, in capsules.

### DETAILED DESCRIPTION OF PREFERRED EMBODIMENTS

A most preferred formulation of the present invention useful for managing blood glucose levels generally, and thereby controlling blood glucose levels in women with polycystic ovarian syndrome (PCOS) so as to alleviate symptoms of PCOS and/or increase fertility in such women, includes alpha lipoic acid (herein “α-lipoic acid”), linolenic acid complex, biotin and coenzyme Q-10. Acceptable ranges of the four constituents per day of the preferred formulation of the present invention are as follows:
α-lipoic acid-200 to 2500 milligrams (“mg.”);
linolenic acid complex-25 to 4000 mg.;
biotin-5 to 25 mg.; and
coenzyme Q-10-50 to 500 mg.

The α-lipoic acid component of the preferred formations of the present invention, is an antioxidant co-enzyme. One form of α-lipoic acid acceptable for use in the formulations of the present invention is a 600 mg. softgel available from Nature's Life® of Larkspur, Calif.

The “linolenic acid complex” component of the preferred formulations as defined herein contains one or more of the following constituents: palmitic, stearic, oleic, linoleic, gamma linolenic, alpha linoleic, icosenoic and erucic acids.

Biotin (C₁₀OH₁₆N₂O₃S) is also known as vitamin B7 or vitamin H. A preferred form of biotin for use in the formulations of the present invention is in 5 mg. capsules.

Coenzyme Q-10 is present in human cells and has a pivotal role in the production of the body's energy, as all ATP is converted to energy with the aid of coenzyme Q-10. A preferred form for use in the formulations of the present invention is a softgel containing 100 mg. ubiquinone.

### EXAMPLE 1

The following daily regimen incorporating the four components of the present invention was developed:
α-lipoic acid—600 mg. tid orally;
linolenic acid complex—1300 mg. bid orally (for a total per day of 25 mg. linolenic acid, 1910 mg. linoleic acid and 130 mg. gamma linolenic acid);
biotin—5 mg. tid orally; and
coenzyme Q-10—100 mg. bid orally.
   The above formulation taken orally with or directly after meals is referred to herein as the Example 1 regimen.

The Example 1 regimen was followed by two adult males previously diagnosed with type 2 diabetes mellitus and being treated with prescription drugs, as described below in Examples 2 and 3.

### EXAMPLE 2

A 59 year old Caucasian male 30 pounds over-weight was first diagnosed with type 2 diabetes mellitus in 1996. Treatment initially began with Metformin and Amaryl®, with dosages increasing over time. The Metformin and Amaryl® dosages were then supplemented with Lantus® injections at bedtime in increasing dosage over the next 3 years, as summarized below in Table A. By November 2006, Lantus® dosage was maximized at 55 units qd, and the patient's endocrinologist was recommending adding a fast-acting insulin at mealtime.

TABLE A

| DATES-all dates | METFORMIN | | METFORMIN | |
|---|---|---|---|---|
| approximate | dosage | | per/day | Other |
| 1996 to 1997 | 850 | mg. bid | 1700 mg. | Amaryl ® 4 mg. qd |
| 1997 to 1998 | 850 | mg. bid | 1700 mg. | Amaryl ® mg. qd |
| | | | | Starlix ® 120 mg. tid |
| | | | | (discontinued after 90 days) |
| 1998 to 1999 | 850 | mg. bid | 1700 mg. | Amaryl ® 8 mg. qd |
| 1999 to 2002 | 850 | mg. tid | 2550 mg. | Amaryl ® 8 mg. qd |
| 2002 to 2004 | 850 | mg. tid | 2550 mg. | Amaryl ® 8 mg. qd |
| | | | | Lantus ® 16 units at bedtime |
| | | | | increased over three years to 55 units |
| | | | | at bedtime |
| 2004 thru | 850 | mg. tid | 2550 mg. | Amaryl ® 8 mg. qd |
| first 3 weeks of | | | | Lantus ® 55 units at bedtime |
| NOV 2006 | | | | |
| NOV 2006 | 850 | mg. tid | 2550 mg. | Amaryl ® 8 mg. qd |
| for 2 days | | | | Lantus ® 48 units at bedtime |
| | | | | Example 1 regimen |
| NOV 2006 to | 850 | mg. tid | 2550 mg. | Amaryl ® 8 mg. qd |
| DEC 2006 for | | | | Lantus ® decreased from 48 |
| 8-10 days | | | | units to 35 units at bedtime |
| | | | | Example 1 regimen |
| DEC 2006 for | 850 | mg. tid | 2550 mg. | Amaryl ® 8 mg. qd |
| next 8-10 days | | | | Lantus ® decreased from 35 |
| | | | | units to 25 units at bedtime |
| | | | | Example 1 regimen |
| DEC 2006 for | 850 | mg. tid | 2550 mg. | Amaryl ® 8 mg. qd |
| next 7 days | | | | Lantus ® 25 units at bedtime |
| | | | | Example 1 regimen |
| JAN 2007 thru | 850 | mg. tid | 2550 mg. | Amaryl ® 4 mg. qd |
| MAY 2007 | | | | Lantus ® 25 units at bedtime |
| | | | | Example 1 regimen |
| JUN 2007 thru | 1000 | mg. bid | 2000 mg. | Amaryl ® 4 mg. qd |
| JULY 2007 | | | | Lantus ® 25 units at bedtime |
| | | | | Example 1 regimen |

During the last week of November 2006, the individual supplemented his prescription drug regimen with the Example 1 regimen taken with or directly after meals with all amounts as described in Example 1, except that a liquid coenzyme Q-10 was not precisely measured and was estimated to range from 100 to 150 mg. per day until April 2007, when 100 mg. softgels were substituted. After two days of the Example 1 regimen, the individual's blood glucose level was substantially lower, and he decreased his Lantus® injections from 55 to 45 units. During the next 8-10 days, while maintaining the Example 1 regimen, his blood glucose levels continued to decrease such that he was able to decrease his Lantus® injections in a step-wise fashion over this time period from 45 to 35 units. During the next 7 days, while continuing to maintain the Example 1 regimen, the individual decreased his Lantus® injections from 35 to 25 units at bedtime. In January 2007, the individual was able to decrease his Amaryl® dosage from 8 mg. per day to 4 mg. per day. In June 2007, the dosage of Metformin was decreased from 2550 mg. per day to 2000 mg. per day, while still maintaining acceptable blood glucose levels.

### EXAMPLE 3

A 58 year old Caucasian male 70 pounds overweight was first diagnosed with type 2 diabetes mellitus in 1999, after which treatment with Metformin, Avandia® and Byetta® progressed as is summarized in Table B below.

TABLE B

| DATES-all | METFORMIN | | METFORMIN | |
|---|---|---|---|---|
| dates approx. | dosage | | per/day | Other |
| 1999 to 2001 | 500 | mg. bid | 1000 mg. | |
| 2001 to 2003 | 500 | mg. bid | 1000 mg. | Avandia ® 4 mg. qd |
| 2003 to 2005 | 500 | mg. tid | 1500 mg. | Avandia ® 4 mg. qd |
| 2005 thru | 1000 | mg. bid | 2000 mg. | Avandia ® 4 mg. qd |
| NOV 2006 | | | | Byetta ® 10 μg. bid |
| DEC 2006 | 1000 | mg. bid | 2000 mg. | Avandia ® 4 mg. qd |
| | | | | Byetta ® 10 μg. bid |
| | | | | Example 1 regimen |
| JAN 2007 thru | 500 | mg. bid | 1000 mg. | Byetta ® 10 μg. bid |
| MAR 2007 | | | | Avandia ® 4 mg. qd |
| | | | | Example 1 regimen |
| APR 2007 thru | 500 | mg. qd | 500 mg. | Byetta ® 10 μg. bid |
| JUL 2007 | | | | Example 1 regimen |

As can be seen above, the individual's prescription drug regimen was increasing in dosage of Metformin over the years, and upon supplementing the prescription drug program with the Example 1 regimen, over time the individual was able to omit the Avandia® and reduce the Metformin dosage to a minimal level.

Without knowing the precise mechanism(s) by which the formulations of the present invention contribute to the maintenance of acceptable blood glucose levels in individuals with type 2 diabetes mellitus while decreasing dosages of Metformin and other prescription drugs which otherwise over time were requiring increased dosages, it is believed that the components of the present invention work synergistically to normalize insulin receptors damaged by the presence of excess insulin in the body. The excess insulin, which is believed over time to cause a trend of steadily increasing down-regulation of the insulin receptors, is at least partially reversed when the formulations of the present invention are orally administered.

While the components of the Example 1 formulation were administered above periodically during the day, orally, in individual softgels and capsules for each component, and so the components may be purchased individually, a most preferred form for administration of the formulations of the present invention is a mixture wherein one or more, and preferably all four, and most preferably three of the components are separately microencapsulated and then packaged together for oral administration in capsules or other forms. In the most preferable form, the alpha-lipoic, coenzyme Q-10 and biotin are micro encapsulated and the linolenic acid complex becomes the matrix in which the microencapsulated components are embedded. Microencapsulation processes are well known to those of skill in the art, but have not been used to package medical foods/nutritional supplements for use as described herein.

When administering mixtures of the separately microencapsulated components of the medical foods and/or nutritional supplements of the present invention, preferred recommended dosages are 5% to 95% of each of the constituents described above. Most preferred dosages are from 50% to 75% of each of the constituents described above. These substantially decreased dosages result from controlled and sustained delivery of the active substances achieved by the use of microcapsules, so that substantially more of each component of the formulations of the present invention reaches the blood circulation. Most preferred ranges of the four constituents per day of the formulation of the present invention when one or more are of the constituents are microencapsulated are as follows:
α-lipoic acid-50 to 1875 milligrams (“mg.”);
linolenic acid complex-12.5 to 3000 mg.;
biotin-2.5 to 18.75 mg.; and
coenzyme Q-10—25 to 375 mg.

Another preferred delivery form of the formulations of the present invention is packaged as a mixture, preferably microencapsulated, in small impermeable, disposable packages such as packets (e.g., 1½×2″ in size) or small tubes (e.g., ¼ diameter×2″ in length) of foil, plastic, or other disposable material. In these configurations, the contents of the packages containing the formulations are mixed with food or a cold liquid.

Alternate formulations and regimens of the present invention include α-lipoic acid, linolenic acid complex, biotin and coenzyme Q-10 and also thiamine, often referred to as vitamin B1. Recommended thiamine dosages to be combined with the formulations of the present invention are from 5 to 25 mg. per day. It is further contemplated that vitamin B12 could be substituted for the thiamine, in dosages of from 20 to 60 μg. per day. In yet another embodiment, a B vitamin complex is combined with the formulations of the present invention. Other formulations and regimens of the present invention include α-lipoic acid, linolenic acid complex, biotin and coenzyme Q-10 and also L-carnatine.

It is contemplated that further components described above may be combined in the formulations of the present invention or administered in conjunction therewith, a further embodiment of the present invention consists essentially of α-lipoic acid, linolenic acid complex, biotin and coenzyme Q-10. While acceptable ranges of daily dosages are
α-lipoic acid-200 to 2500 mg.;
linolenic acid complex-25 to 4000 mg.;
biotin-5 to 25 mg.; and
coenzyme Q-10-50 to 500 mg,
   any of the other formulations described herein may be limited to consist essentially of the stated ingredients at the stated ingredient dosages or dosage ranges.

In another embodiment of the method of the present invention, a formulation of the present invention is ingested by a woman with PCOS. Acceptable daily dosages of the constituents in the formulations of the present invention are expected to have lower acceptable amounts in some women, reflecting the smaller average size of women as compared to the average size of men, and the possibility imbalances in insulin levels and insulin receptor activity in women with PCOS may not be result from hyperinsulinemia caused by excess production by pancreatic beta cells. Presently preferred ranges of acceptable daily dosages for women with PCOS are:
α-lipoic acid-100 to 2500 mg.;
linolenic acid complex-10 to 4000 mg.;
biotin-2 to 25 mg.; and
coenzyme Q-10—25 to 500 mg,

In women with PCOS ingesting such amounts, as management of blood glucose levels, insulin levels and/or insulin receptivity is achieved, symptoms associated with PCOS are expected to decrease. When fertility issues are present, fertility is expected to increase.

The above method and formulation of the present invention applicable to women with PCOS, when ingested by a male or female adult, is expected to obviate development or progression of Alzheimer's disease. By ingesting the formulations of the present invention, either as a single capsule or as a combination of constituents taken separately but in conjunction with each other, brain insulin levels and/or insulin receptor functionality is expected to improve.

While there have been described above the principles of the present invention in conjunction with preferred embodiments thereof, it is to be clearly understood that the foregoing description is made only by way of example and not as a limitation to the scope of the invention. Particularly, it is recognized that the teachings of the foregoing disclosure will suggest other modifications to those persons skilled in the relevant art. Such modifications may involve other features which are already known and which may be used instead of or in addition to features already described herein. Although claims have been formulated in this application to particular combinations of features, it should be understood that the scope of the disclosure herein also includes any novel feature or any novel combination of features disclosed either explicitly or implicitly or any generalization or modification thereof which would be apparent to persons skilled in the relevant art, whether or not such relates to the same invention as presently claimed in any claim and whether or not it mitigates any or all of the same technical problems as confronted by the present invention. The applicants hereby reserve the right to formulate new claims to such features and/or combinations of such features during the prosecution of the present application or of any further application derived therefrom.

1. A composition for oral administration in women for decreasing severity and/or presence of symptoms associated with polycystic ovarian syndrome (PCOS), comprising:
α-lipoic acid;
linolenic acid complex;
biotin; and
coenzyme Q-10.

2. The composition of claim 1 wherein a daily dosage comprises:
α-lipoic acid-100 to 2500 mg.;
linolenic acid complex-10 to 4000 mg.;
biotin—2 to 25 mg.; and
coenzyme Q-10—25 to 500 mg.

3. The composition of claim 2, wherein the linolenic acid complex includes gamma linolenic acid.

4. A method of managing blood glucose levels, insulin levels and/or insulin receptivity in a woman with polycystic ovarian syndrome (PCOS) and thereby decreasing severity and/or presence of one or more symptom associated with PCOS, comprising ingesting at least once a day an effective amount of four constituents, separately or together in any combination, comprising α-lipoic acid, linolenic acid complex, biotin,.and coenzyme Q-10.

5. The method of claim 4, wherein a daily dosage of the composition comprises:
α-lipoic acid—100 to 2500 mg.;
linolenic acid complex—10 to 4000 mg.;
biotin—2 to 25 mg.; and
coenzyme Q-10—25 to 500 mg.

6. A method of maintaining or improving levels of insulin and/or insulin receptor functionality in the brain, comprising administering to an adult at least the following constituents separately or together: α-lipoic acid, linolenic acid complex, biotin, and coenzyme Q-10.

7. The method of claim 6, wherein a daily dosage comprises:
α-lipoic acid—100 to 2500 mg.;
linolenic acid complex—10 to 4000 mg.;
biotin—2 to 25 mg.; and
coenzyme Q-10—25 to 500 mg.

8. The method of claim 7, wherein the linolenic acid complex includes gamma linolenic acid.
